(19) United States
(12) Patent Application Publication
(10) Pub. No.: US 2006/0140890 A1
(43) Pub. Date: Jun. 29, 2006

(54) Oily solid cosmetic and method for production thereof

(76) Inventors: Atsushi Miida, Saitama (JP); Toshiyuki Ito, Saitama (JP); Junko Nakayama, Tokyo (JP); Soichiro Watanabe, Tokyo (JP); Tsuyoshi Hata, Saitama (JP)

Correspondence Address: GREENBLUM & BERNSTEIN, P.L.C., 1950 ROLAND CLARKE PLACE, RESTON, VA, 20191, US

(21) Appl. No.: 10/528,190

(22) PCT Filed: Oct. 9, 2003
(86) PCT No.: PCT/JP03/12971

§ 371 Date: Jan. 23, 2006

(30) Foreign Application Priority Data
Oct. 11, 2002 (JP) 2002-298346

(51) Int. Cl. A61K 8/81

(52) U.S. Cl. 424/64

(57) ABSTRACT

An oil-based solid cosmetic comprising (A) vitamin A and/or a fatty acid ester thereof, (B) silicic anhydride, (C) an oil-soluble antioxidant, and (D) an oily ingredient existing as paste and/or liquid at room temperature is disclosed. The aforementioned oil-based solid cosmetic, wherein the ingredient (B) is aerosol silicic anhydride, and the aforementioned oil-based solid cosmetic, wherein the ingredient (D) is polybutene and/or heavy liquid isoparaffin are also disclosed.

### TECHNICAL FIELD

The present invention relates to an oil-based solid cosmetic which can be stably blended with vitamin A and/or a fatty acid ester thereof, has favorable properties for use, and exhibits favorable feelings upon use, and a method for producing the same.

### BACKGROUND ART

It is known that vitamin A and/or a fatty acid ester thereof is an ingredient effective for improvement of rough skin, prophylactic or therapeutic treatment of skin keratosis etc., further, prevention and recovery of aging of skin, and the like. However, vitamin A and/or a fatty acid ester are structurally very unstable, and they may easily cause isomerization, decomposition, polymerization etc. with light, air, heat, metal ions, and the like. Therefore, it is difficult to stably blend it in cosmetics. In order to solve the aforementioned problem, there have been made approaches such as blending of antioxidant or metal ion sequestering agent, and blending of a lot of oil ingredient.

For example, Japanese Patent Unexamined Publication (KOKAI) No. 6-32713 describes that an external preparation for skin improved in stability of vitamin A and/or a fatty acid ester thereof is obtained by blending a particular oil-soluble antioxidant, ethylenediaminetetraacetate, and a benzophenone type compound together with vitamin A and/or the fatty acid ester thereof, and describes examples of oil essence or oil gel in Examples 6, 8, and 9.

Further, Japanese Patent Unexamined Publication No. 6-32720 describes that an external preparation for skin improved in stability of vitamin A and/or a fatty acid ester thereof is obtained by blending a pentaerythritol fatty acid ester and/or trimethylolpropane together with vitamin A and/or the fatty acid ester thereof, and describes examples of oil essence in Examples 9 and 11.

Furthermore, concerning preservation of vitamin A, “Kagaku Dai-Jiten (Great Dictionary of Chemistry)”, ed by Oki, M. et al. (published by Tokyo Kagaku Dojin on Oct. 20, 1989) contains information that vitamin A is converted into an ester such as palmitate, dissolved in fresh vegetable oil, and added with an antioxidant (d1-α-tocopherol, dibutylhydroxytoluene, etc.) for preservation (p. 1870, right column, section of ““Vitamin A”).

In addition, Japanese Patent Unexamined Publication No. 6-24956 describes an external preparation for skin blended with vitamin A and a polyoxyalkylene-modified organopolysiloxane as an external preparation for skin capable of improving a rough skin, and an example of the use thereof for lipstick in Example 9.

However, all the aforementioned documents do not describe nor suggest the combination, effect, and formulation of the present invention.

### DISCLOSURE OF THE INVENTION

Conventional oil-based liquid type and gel type formulations are inconvenient for taking out from a bottle or tube to hand or cotton, and show inferior properties for use and feelings upon use such as lack of smoothness and adhesion to skin upon use. Therefore, an object of the present invention is to provide a cosmetic stably containing vitamin A and/or a fatty acid ester thereof and showing superior properties for use as well as smoothness and adhesion to skin upon use.

The inventors of the present invention conducted various researches under the aforementioned factual situation, and as a result, they found that the aforementioned inconvenience upon use and feelings upon use could be improved by preparing a cosmetic containing vitamin A and/or a fatty acid ester thereof as an oil-based solid cosmetic, especially a stick-shaped oil-based solid cosmetic, containing an oily ingredient existing as paste and/or liquid at room temperature and silicic anhydride. They further found that stability of vitamin A and/or a fatty acid ester thereof could be improved by blending with an oil-soluble antioxidant, and the cosmetic exhibiting the lasting effect thereof could be obtained.

The oil-based solid cosmetic of the present invention thus is an oil-based solid cosmetic comprising the following ingredients (A) to (D):

(A) Vitamin A and/or a fatty acid ester thereof

(B) Silicic anhydride

(C) Oil-soluble antioxidant

(D) Oily ingredient existing as paste and/or liquid at room temperature.

Other embodiments of the present invention include the aforementioned oil-based solid cosmetic, wherein the ingredient (B) is aerosol silicic anhydride, and the ingredient (D) is polybutene and/or heavy liquid isoparaffin; the aforementioned oil-based solid cosmetic, which does not substantially contain moisture; the aforementioned oil-based solid cosmetic, which is in the shape of a stick; and the aforementioned oil-based solid cosmetic, which is a lip stick, lip gloss, or lip cream.

In another aspect, the present invention provides a process for producing an oil-based solid cosmetic, comprising preparing a composition containing (A) vitamin A and/or a fatty acid ester thereof, (B) silicic anhydride, (C) an oil-soluble antioxidant, and (D) an oily ingredient existing as paste and/or liquid at room temperature; the aforementioned process comprising molding the composition into a stick shape; and the aforementioned process, comprising filling a container with the composition.

### BEST MODE FOR CARRYING OUT THE INVENTION

Hereafter, embodiments of the present invention will be explained in detail.

The ingredient (A), vitamin A and/or a fatty acid ester thereof, used for the present invention is an ingredient effective for improvement of rough skin, prophylactic or therapeutic treatment of skin keratosis, prevention or recovery of skin aging, and the like. Examples of the vitamin A and/or fatty acid ester thereof used for the present invention include vitamin A, vitamin A acetic acid ester, vitamin A palmitic acid ester, and the like, and those of all trans type and 13-cis type are preferred. A mixture thereof may also be used. One or more kinds of them may optionally be combined for use.

Although the content of the vitamin A and/or fatty acid ester thereof as the ingredient (A) in the oil-based solid cosmetic of the present invention is not particularly limited, it is preferably 0.001 to 10 mass % (hereafter simply indicated as “%”), and from the point of superior effects for improvement of rough skin, prophylactic or therapeutic treatment of skin keratosis, prevention or recovery of skin aging, and the like, and feelings upon use such as tackiness to skin, in particular, it is more preferably 0.01 to 1%.

The ingredient (B), silicic anhydride, used for the present invention is added for the purposes of maintaining the form of the composition as solid and improving properties for use, as well as improving feelings upon use and storage stability of the composition. As the ingredient (B), although silicic anhydride of any shape such as those of aerosol, porous shape, non-porous shape and spherical shape may be used so long as silicic anhydride usually used for cosmetics is chosen, aerosol silicic anhydride is particularly preferred. Examples of the aerosol silicic anhydride include, for example, those obtainable by hydrolysis of silicon tetrachloride in a hydrogen and oxygen flame, and examples of commercial product thereof include AEROSIL 50, AEROSIL 130, AEROSIL 200, AEROSIL 200V, AEROSIL 200CF, AEROSIL 200FAD, AEROSIL 300, AEROSIL 300CF, AEROSIL 380 (those are produced by Nippon Aerosil Co., Ltd.), and the like. Further, hydrophobic aerosol silicic anhydride obtained by treating the aforementioned aerosol silicic anhydride with a reactive alkylsilane, reactive organosilazane or the like may also be used. Examples of the method for the hydrophobization treatment (also referred to as “lipophilization treatment”) include dimethylsilylation treatment with dimethyldichlorosilane, trimethylsilylation treatment with trimethylchlorosilane or hexamethyldisilazane, octylsilylation treatment with octyltrichlorosilane, siliconization treatment with dimethylpolysiloxane or methyl hydrogen polysiloxane, coating treatment with a metal soap compound, and the like. Examples of commercial products of hydrophobized silicic anhydride include AEROSIL R-972, AEROSIL R974, and AEROSIL R976 (these are treated with dimethyldichlorosilane), AEROSIL RX200, AEROSIL RX300 (these are treated with hexamethyldisilazane), AEROSIL R805 (treated with octylsilane), AEROSIL RY200, AEROSIL RY300 (these are treated with dimethylsiloxane) (all produced by Nippon Aerosil Co., Ltd.), Cabosil TS530 (treated with trimethylchlorosilane, produced by Cabot Corp.), and the like.

The ingredient (B), silicic anhydride, used for the present invention preferably has a mean primary particle diameter of 50 nm or less, more preferably 7 to 20 nm. The mean primary particle diameter of the aerosol silicic anhydride is usually 50 nm or less.

As the ingredient (B), silicic anhydride, used for the present invention, one or more kinds of silicic anhydride may be used as required, and the content thereof is preferably 0.01 to 30%, more preferably 0.5 to 20%, in view of maintenance of the form, feelings upon use and storage stability.

The ingredient (C), an oil-soluble antioxidant, used for the present invention contribute to prevention of degradation of the ingredient (A), vitamin A and/or a fatty acid ester thereof, over time, prevention of change of odor and discoloration accompanying the degradation, and improvement of storage stability. The oil-soluble antioxidant as the ingredient (C) used for the present invention is not particularly limited so long as an oil-soluble antioxidant usually used for cosmetics is chosen, and examples thereof include dibutylhydroxytoluene (hereinafter referred to as “BHT”), butylhydroxyanisole (hereinafter referred to as “BHA”), α-, β-, γ- and σ-tocopherols, propyl gallate, L-ascorbic acid fatty acid esters, and the like. One or more kinds of these may be appropriately chosen and combined for use.

Although the content of the oil-soluble antioxidant as the ingredient (C) in the cosmetic of the present invention is not particularly limited, it is preferably 0.001 to 10%, and from the point of superior effect for improvement of storage stability of vitamin A and/or a fatty acid ester thereof, and feelings upon use such as tackiness to skin, in particular, it is more preferably 0.01 to 1%.

The ingredient (D), an oily ingredient existing as paste and/or liquid at room temperature, used for the present invention contributes to improvement of smoothness and adhesion to skin upon use, and improvement of storage stability of vitamin A and/or a fatty acid ester thereof. Any oily ingredients generally used for cosmetics may be used. The origin is not particularly limited, and animal oils, vegetable oils, synthetic oils, and the like may be used. Examples include hydrocarbons, oils and fats, waxes, hydrogenated oils, ester oils, aliphatic acids, higher alcohols, silicone oils, fluorine type oils, lanolin derivatives, and the like. Specific examples include hydrocarbons such as liquid paraffins, heavy liquid isoparaffins, α-olefin oligomers, squalane, petrolatum, polyisobutylenes, and polybutenes, oils and fats such as olive oil, castor oil, mink oil, and macadamia nut oil, Jojoba oil, esters such as diisostearoyl malate, cetyl isooctanate, isopropyl myristate, isopropyl palmitate, octyldodecyl myristate, polyglyceryl diisostearate, digryceryl triisostearate, neopentylglycol dioctanoate, cholesterol fatty acid esters and di(cholesteryl/behenyl/octyldodecyl) N-lauroyl-L-glutamate, aliphatic acids such as isostearic acid and oleic acid, higher alcohols such as oleyl alcohol and isostearyl alcohol, silicones such as low polymerization degree dimethylpolysiloxanes, high polymerization degree dimethylpolysiloxanes, methylphenylpolysiloxanes, decamethylcyclopentasiloxane, octamethylcyclotetrasiloxane, polyether-modified polysiloxanes, polyoxyalkylene/alkylmethylpolysiloxane/methylpolysiloxane copolymers, alkoxyl-modified polysiloxanes, and fluorine-modified polysiloxanes, fluorine type oils such as perfluorodecane, perfluorooctane and perfluoropolyethers, lanolin, lanolin derivatives such as lanolin acetate, lanolin aliphatic acid isopropyl esters, and lanolin alcohols, and the like. Among these, polybutenes and heavy liquid isoparaffins are particularly preferred in view of feelings upon use such as adhesion to skin, and examples of commercial products thereof include Parleam 16, Parleam 18 (these are produced by Nippon Oil & Fats Co., Ltd.), Polybutene 100R, Polybutene 300R, Polybutene 2000H (these are produced by Idemitsu Petrochemical Co., Ltd.), and the like. One or more kinds of these can be used as required.

Although the content of the ingredient (D), an oily ingredient existing as paste and/or liquid at room temperature, used for the present invention is not particularly limited, it is preferably 5 to 99%, more preferably 10 to 80%. The content within such a range is preferred in view of feelings upon use and storage stability.

Because of inclusion of the aforementioned ingredients (A) to (D), the oil-based solid cosmetic of the present invention can be an oil-based solid cosmetic that can be stably blended with vitamin A and/or a fatty acid ester thereof and maintain the form, and shows favorable properties for use and feelings upon use.

The meaning of the term “solid” used in the present specification should be construed in its broadest sense, and soft solids and semi-solids are also included so long as they do not show fluidity at room temperature (about 25□), but maintain a certain shape.

Examples of the formulation type of the oil-based solid cosmetic of the present invention include oil type and emulsified type (W/O type), and examples of the shape include various shapes, for example, shapes of soft solids filled in plate-like containers, solid stick shapes, and the like. Further, the oil-based solid cosmetic that does not substantially contain moisture is more preferred in view of improvement of stability of vitamin A and/or a fatty acid ester thereof, and as for the shape, those of stick shape are more preferred in view of convenience upon use, feelings upon use such as smoothness, less tackiness, and adhesion to skin, and storage stability of vitamin A and/or a fatty acid ester thereof and the preparation. In particular, the oil-based solid cosmetic in a stick shape is suitable as a cosmetic for topical application, for example, application to lip, eyelid, eye area or the like alone.

If necessary, in order to give various effects, various ingredients other than the essential ingredients, such as oily ingredients, powdery ingredients, surfactants, ultraviolet absorbers, fading inhibitors, preservatives, chelating agents, water-soluble polymers, film-forming ingredients, moisturizers, cosmetic ingredients, and fragrances may be blended in the oil-based solid cosmetic of the present invention to such a degree that they should not lower the advantages of the present invention.

### EXAMPLES

Hereafter, the present invention will be explained more specifically by referring to examples. However, the present invention is not limited by the examples at all.

### Examples 1 to 9 and Comparative Examples 1 to 3

Oil-based solid cosmetics (stick shape) containing vitamin A palmitate and having the compositions shown in Table 1 (examples) and Table 2 (comparative examples) were prepared, and evaluated by the following methods for skin effect (improvement of rough skin), property for use (ease of use), feelings upon use (smoothness and adhesion upon use) and storage stability (evaluation of discoloration and change of odor, evaluation of separation property (sweating)). The results are also shown in Tables 1 and 2.

TABLE 1
(%)

| | | Examples | | | | | | | | |
|---|---|---|---|---|---|---|---|---|---|---|
| No. | Ingredients | 1 | 2 | 3 | 4 | 5 | 6 | 7 | 8 | 9 |
| 1 | Vitamin A palmitic acid ester | 0.25 | 0.001 | 4 | 0.25 | 0.25 | 0.25 | 0.25 | 0.25 | 0.25 |
| 2 | Lipophilized silicic anhydride (Note 1) | 1 | 1 | 1 | 0.1 | 5 | 1 | 1 | 0.5 | 0.5 |
| 3 | BHT | 0.1 | 0.1 | 0.1 | 0.1 | 0.1 | 0.001 | 1 | 0.1 | 0.1 |
| 4 | Heavy liquid isoparaffin (Note 2) | 10 | 10 | 10 | 10 | 10 | 10 | 10 | 5 | 20 |
| 5 | Polyethylene/microcrystalline | 6.5 | 6.5 | 6.5 | 6.5 | 6.5 | 6.5 | 6.5 | 6.5 | 6.5 |
| | wax mixture | | | | | | | | | |
| 6 | Polyethylene wax | 2 | 2 | 2 | 2 | 2 | 2 | 2 | 2 | 2 |
| 7 | Ceresin wax | 6 | 6 | 6 | 6 | 6 | 6 | 6 | 6 | 6 |
| 8 | Paraffin | 10 | 10 | 10 | 10 | 10 | 10 | 10 | 10 | 10 |
| 9 | Liquid paraffin | balance | balance | balance | balance | balance | balance | balance | balance | balance |
| 10 | Nylon powder 1 (Note 3) | 4 | 4 | 4 | 4 | 4 | 4 | 4 | 4 | 4 |
| 11 | Nylon powder 2 (Note 4) | 3 | 3 | 3 | 3 | 3 | 3 | 3 | 3 | 3 |
| 12 | Talc | 7 | 7 | 7 | 7 | 7 | 7 | 7 | 7 | 7 |
| 13 | Disodium | 0.1 | 0.1 | 0.1 | 0.1 | 0.1 | 0.1 | 0.1 | 0.1 | 0.1 |
| | ethylenediaminetetraacetate | | | | | | | | | |
| 14 | Titanium oxide | 1.5 | 1.5 | 1.5 | 1.5 | 1.5 | 1.5 | 1.5 | 1.5 | 1.5 |

Evaluation items and judgment results

| | | | | | | | | | |
|---|---|---|---|---|---|---|---|---|---|
| Skin effect: Rough skin improving effect | ⊚ | ◯ | ⊚ | ⊚ | ⊚ | ◯ | ⊚ | ⊚ | ⊚ |
| Property for use: Ease of use | ⊚ | ⊚ | ⊚ | ⊚ | ◯ | ⊚ | ⊚ | ⊚ | ⊚ |
| Feelings upon use: | | | | | | | | | |
| Smoothness upon use | ⊚ | ⊚ | ⊚ | ⊚ | ◯ | ⊚ | ⊚ | ⊚ | ⊚ |
| Adhesion | ⊚ | ◯ | ⊚ | ⊚ | ⊚ | ⊚ | ⊚ | ⊚ | ⊚ |
| Storage stability: | | | | | | | | | |
| Evaluation of discoloration and | ⊚ | ⊚ | ◯ | ⊚ | ⊚ | ◯ | ◯ | ⊚ | ⊚ |
| change of odor | | | | | | | | | |
| Evaluation of separation | ⊚ | ⊚ | ⊚ | ◯ | ⊚ | ⊚ | ⊚ | ◯ | ◯ |
| property | | | | | | | | | |

Note 1: AEROSIL R-972 (produced by Nippon Aerosil, mean particle diameter of primary particles: 16 nm)
Note 2: Parleam 18 (produced by Nippon Oil & Fats Co., Ltd.)
Note 3: Toray Nylon Powder SP-500 (produced by Toray Industries, Inc.)
Note 4: Rilsan Powder S (produced by Rilsan, France)

TABLE 2
(%)

| | | Comparative Examples | | |
|---|---|---|---|---|
| No. | Ingredients | 1 | 2 | 3 |
| 1 | Vitamin A palmitic acid ester | — | 0.25 | 0.25 |
| 2 | Lipophilized silicic anhydride | 1 | — | 1 |
| | (Note 1) | | | |
| 3 | BHT | 0.1 | 0.1 | — |
| 4 | Heavy liquid isoparaffin | 10 | 10 | 10 |
| | (Note 2) | | | |
| 5 | Polyethylene/microcrystalline | 6.5 | 6.5 | 6.5 |
| | wax mixture | | | |
| 6 | Polyethylene wax | 2 | 2 | 2 |
| 7 | Ceresin wax | 6 | 6 | 6 |
| 8 | Paraffin | 10 | 10 | 10 |
| 9 | Liquid paraffin | balance | balance | balance |
| 10 | Nylon powder 1 (Note 3) | 4 | 4 | 4 |
| 11 | Nylon powder 2 (Note 4) | 3 | 3 | 3 |
| 12 | Talc | 7 | 7 | 7 |
| 13 | Disodium | 0.1 | 0.1 | 0.1 |
| | ethylenediaminetetraacetate | | | |
| 14 | Titanium oxide | 1.5 | 1.5 | 1.5 |

| | | | |
|---|---|---|---|
| Evaluation items and judgment results | | | |
| Skin effect: | X | X | ⊚ |
| Rough skin improving effect | | | |
| Property for use: Ease of use | ⊚ | ⊚ | ⊚ |
| Feelings upon use: | | | |
| Smoothness upon use | ⊚ | ⊚ | ⊚ |
| Adhesion | ⊚ | ⊚ | ⊚ |
| Storage stability: | | | |
| Evaluation of discoloration and | ⊚ | ⊚ | X |
| Evaluation of separation property | ⊚ | X | ⊚ |

Note 1: AEROSIL R-972 (produced by Nippon Aerosil, mean particle diameter of primary particles: 16 nm
Note 2: Parleam 18 (produced by Nippon Oil & Fats Co., Ltd.)
Note 3: Toray Nylon Powder SP-500 (produced by Toray Industries, Inc.)
Note 4: Rilsan Powder S (produced by Rilsan, France)

(Production Method)

The ingredients (1) and (3) to (9) were uniformly heated and mixed, then added with the ingredient (2) and (10) to (14), and uniformly mixed. This mixture was filled in a container and solidified by cooling to obtain a product.

(Evaluation Method for Skin Effect)

A use test was conducted for each sample by a panel of 20 experts (application after face washing in morning and evening for 1 month) to evaluate rough skin improving effect according to the following 7-stage evaluation criteria. From the total of the evaluation scores of the members of the panel, an average was calculated and judged according to the following 4-stage judgment criteria.

(Evaluation Method for Property for Use and Feelings Upon Use)

A use test was conducted for each sample by a panel of 20 experts (application after face washing) to confirm a property for use (ease of use) and evaluate smoothness and adhesion to skin upon use according to the following 7-stage evaluation criteria. From the total of the evaluation scores of the members of the panel, an average was calculated and judged according to the following 4-stage judgment criteria.

(7-Stage Evaluation Criteria for Skin Effect, Property for Use and Feelings Upon Use)

| Evaluation score: | Evaluation |
|---|---|
| 6 Points: | Very good |
| 5 Points: | Good |
| 4 Points: | Fairly good |
| 3 Points: | Average |
| 2 Points: | Fairly bad |
| 1 Point: | Bad |
| 0: | Extremely bad |

(4-Stage Judgment Criteria for Skin Effect, Property for Use and Feelings Upon Use)
More than 5 points: Extremely good (⊚)
More than 3 points and not more than 5 points: Good (◯)
More than 2 points and not more than 3 points: Fairly bad (Δ)
Not more than 2 points: Bad (X)
(Storage Stability: Test Method for Change of Odor and Discoloration)

Each sample was stored in an incubator at 40° C. or 5° C. for one month, and the condition was judged on the basis of the condition immediately after preparation according to the following 4-stage judgment criteria (for storage stability).

(Storage Stability: Test Method for Separation Property (Sweating))

Each sample was stored at 45° C. for two weeks, and the condition was judged and compared with that of the sample stored at room temperature to make a judgment according to the following 4-stage judgment criteria (for storage stability).

(4-Stage Criteria for Storage Stability:)

⊚: Good condition with no change

◯: Almost no change, but slight change

Δ: Considerable change

X: Bad condition with change

As clearly seen from the results shown in Tables 1 and 2, the oil-based solid cosmetics of Examples 1 to 9 according to the present invention showed superior skin effect, feelings upon use and storage stability compared with the oil-based solid cosmetics of Comparative Examples 1 to 3.

### Example 10

### Lip Cream (Metal Plate-Filled Type)

| (Ingredients) | (%) |
|---|---|
| 1. Polyethylene/microcrystalline wax mixture | 2.5 |
| 2. Polyethylene wax | 2.0 |
| 3. Petrolatum | 20.0 |
| 4. Glyceryl tri-2-ethylhexanoate | 20.0 |
| 5. Squalane | 5.0 |
| 6. Liquid paraffin | balance |
| 7. Heavy liquid isoparaffin (Note 1) | 20.0 |
| 8. Dimethyldichlorosilane-treated silicic anhydride (Note 2) | 10.0 |
| 9. 1,3-Butylene glycol | 0.5 |
| 10. Sorbitan sesquioleate | 0.1 |
| 11. Acrylic acid/alkyl methacrylate copolymer (Note 3) | 0.01 |
| 12. Aloe vera extract liquid | 0.1 |
| 13. BHT | 0.1 |
| 14. Vitamin A | 1.0 |
| 15. Vitamin A palmitic acid ester | 0.5 |
| 16. Fragrance | 0.05 |

Note 1: Parleam 18 (produced by Nippon Oil & Fats Co., Ltd.)
Note 2: AEROSIL R-974 (produced by Nippon Aerosil Co., Ltd., mean particle diameter of primary particles: 12 nm)
Note 3: Pemulen TR-2 (produced by NOVEON)

(Production Method)

The ingredients (1) to (7) and (13) to (16) were uniformly heated and mixed, then added with the ingredient (8), (9) to (12) and (16), and uniformly mixed. This mixture was filled in a container and solidified to obtain a product.

The lip cream of Example 10 had superior properties for use, and showed good feelings upon use such as smoothness and adhesion upon application, as well as superior skin effect and storage stability.

### Example 11

### Lipstick (Stick Shape)

| (Ingredients) | (%) |
|---|---|
| 1. Glyceryl (behenate/eicosanedicarboxylate) | 5.0 |
| 2. Polyethylene wax | 5.0 |
| 3. Candelilla wax | 5.0 |
| 4. Rosin acid pentaerythritol ester | 2.0 |
| 5. Heavy liquid isoparaffin (Note 1) | 30.0 |
| 6. Polybutene (Note 2) | 5.0 |
| 7. Liquid Paraffin | balance |
| 8. Glyceryl tri-2-ethylhexanoate | 15.0 |
| 9. Silicic anhydride (Note 3) | 0.5 |
| 10. Red No. 202 | Optimum amount |
| 11. Yellow No. 4 | Optimum amount |
| 12. Fragrance | 0.05 |
| 13. BHT | 0.1 |
| 14. Vitamin A palmitic acid ester | 1.0 |

Note 1: Parleam 18 (produced by Nippon Oil & Fats Co., Ltd.)
Note 2: Polybutene 300R (produced by Idemitsu Petrochemical Co., Ltd.)
Note 3: AEROSIL 300 (produced by Nippon Aerosil Co., Ltd., mean particle diameter of primary particles: 7 nm)

(Production Method)

The ingredients (1) to (8), (13) and (14) were uniformly heated and mixed, then added with the ingredient (9) and (10) to (12), and uniformly mixed. This mixture was molded by filling and solidified to obtain a product.

The lipstick of Example 11 in the shape of a stick had superior properties for use, and showed good feelings upon use such as smoothness and adhesion upon application, as well as superior skin effect and storage stability.

### Example 12

### Lipstick (Metal Plate-Filled Type)

| (Ingredients) | (%) |
|---|---|
| 1. Glyceryl (behenate/eicosanedicarboxylate) | 10.0 |
| 2. Polyethylene wax | 3.0 |
| 3. Candelilla wax | 3.0 |
| 4. Rosin acid pentaerythritol ester | 2.0 |
| 5. Heavy liquid isoparaffin (Note 1) | 10.0 |
| 6. Polybutene (Note 2) | 5.0 |
| 7. Liquid paraffin | balance |
| 8. Glyceryl tri-2-ethylhexanoate | 15.0 |
| 9. Dimethyldichlorosilane-treated silicic | 0.5 |
| anhydride (Note 3) | |
| 10. Red iron oxide-coated titanium mica | 15.0 |
| 11. Red No. 202 | Optimum amount |
| 12. Yellow No. 4 | Optimum amount |
| 13. Black iron oxide | Optimum amount |
| 14. Aloe vera extract liquid | 0.1 |
| 15. Tocopherol | 0.05 |
| 16. BHT | 0.1 |
| 17. Vitamin A palmitic acid ester | 1.0 |
| 18. Vitamin A | 0.5 |
| 19. Fragrance | 0.05 |

Note 1: Parleam 18 (produced by Nippon Oil & Fats Co., Ltd.)
Note 2: Polybutene 300R (produced by an Idemitsu Petrochemical Co., Ltd.)
Note 3: AEROSIL R-974 (Produced by Nippon Aerosil Co., Ltd., mean particle diameter of primary particles: 12 nm)

(Production Method)

The ingredients (1) to (8) and (15) to (18) were uniformly heated and mixed, then added with the ingredient (9), (10) to (14) and (19), and uniformly mixed. This mixture was molded by filling and solidified to obtain a product.

The lipstick of Example 12 of the metal plate-filled type had superior properties for use, and showed good feelings upon use such as smoothness and adhesion upon application, as well as superior skin effect and storage stability.

### Example 13

### Lip Gloss

| (Ingredients) | (%) |
|---|---|
| 1. Glyceryl (behenate/eicosanedicarboxylate) | 1.5 |
| 2. Polyethylene wax | 4.5 |
| 3. Candelilla wax | 3.0 |
| 4. Rosin acid pentaerythritol ester | 0.1 |
| 5. Polybutene (Note 1) | 40.0 |
| 6. Diisostearyl malate | 6.0 |
| 7. Liquid paraffin | balance |
| 8. Silicic anhydride (Note 2) | 8.0 |
| 9. Vitamin A | 0.1 |
| 10. BHA | 0.1 |
| 11. Ultraviolet absorber (Note 3) | 0.1 |
| 12. Red No. 202 | Optimum amount |
| 13. Ultramarine blue | Optimum amount |
| 14. Titanium oxide-treated synthetic mica | 15.0 |
| 15. Lame agent (Note 4) | Optimum amount |
| 16. BHT | 0.1 |

Note 1: Polybutene 300R (produced by Idemitsu Petrochemical Co., Ltd.)
Note 2: AEROSIL 300 (produced by Nippon Aerosil Co., Ltd., mean particle diameter of primary particles: 7 nm)
Note 3: Uvinul MC80 (produced by BASF A.G.)
Note 4: Daiya Hologram (produced by DAIYA KOGYO Co., Ltd.)

The ingredients (1) to (7), (9) to (11) and (16) were uniformly heated and mixed, then added with the ingredient (8) and (12) to (15), and uniformly mixed. This mixture was molded by filling and solidified to obtain a product.

The lip gloss of Example 13 had superior properties for use, and showed good feelings upon use such as smoothness and adhesion upon application, as well as superior skin effect and storage stability.

### INDUSTRIAL APPLICABILITY

The oil-based solid cosmetic of the present invention can be stably blended with vitamin A and/or a fatty acid ester thereof, has superior properties for use, and shows superior feelings upon use such as smoothness and adhesion to skin upon use, and superior storage stability.

1. An oil-based solid cosmetic comprising the following ingredients (A) to (D):
(A) vitamin A and/or a fatty acid ester thereof,
(B) silicic anhydride,
(C) an oil-soluble antioxidant, and
(D) an oily ingredient existing as paste and/or liquid at room temperature.

2. The oil-based solid cosmetic according to claim 1, wherein the ingredient (B), silicic anhydride, is aerosol silicic anhydride.

3. The oil-based solid cosmetic according to claim 1, the ingredient (D), oily ingredient existing as paste and/or liquid at room temperature, is polybutene and/or heavy liquid isoparaffin.

4. The oil-based solid cosmetic according to claim 1, which does not substantially contain moisture.

5. The oil-based solid cosmetic according to claim 1, which is in the shape of a stick.

6. The oil-based solid cosmetic according to claim 1, which is a lipstick, lip cream or lip gloss.

7. A method for producing an oil-based solid cosmetic, comprising preparing a composition containing (A) vitamin A and/or a fatty acid ester thereof, (B) silicic anhydride, (C) an oil-soluble antioxidant, and (D) an oily ingredient existing as paste and/or liquid at room temperature.

8. The method according to claim 7, comprising molding the composition into a stick shape.

9. The method according to claim 7, comprising filling a container with the composition.
